(19) United States
(12) Patent Application Publication
(10) Pub. No.: US 2025/0329429 A1
(43) Pub. Date: Oct. 23, 2025

(54) SYSTEMS AND METHODS FOR GENERATING COSMETIC MEDICAL TREATMENT RECOMMENDATIONS VIA MACHINE LEARNING

(71) Applicant: AI AESTHETICS LLC, Simi Valley, CA (US)

(72) Inventors: Alan Darush, Porter Ranch, CA (US); Nils Eric Bernschneider, Frankfurt (DE)

(21) Appl. No.: 19/187,963

(22) Filed: Apr. 23, 2025

Related U.S. Application Data
(60) Provisional application No. 63/637,802, filed on Apr. 23, 2024.

(51) Int. Cl. G16H 20/00; G06T 7/00; G16H 30/40; G16H 50/20; G16H 50/50

(52) U.S. Cl. CPC G16H 20/00; G06T 7/0012; G16H 30/40; G16H 50/20; G16H 50/50; G06T 2207/10004; G06T 2207/20081; G06T 2207/20084; G06T 2207/30088; G06T 2207/30201

(57) ABSTRACT

The method for generating medical treatment recommendations comprises: scanning a relevant part among the face, head, and body of a user; mapping the scanned relevant part onto a mesh model to ensure consistent positioning; positioning the scanned relevant part in the right angle using the mesh model; detecting a set of features associated with the scanned relevant part using the mesh model; determining a treatment area that is needed to be treated based on the detected features associated with the scanned relevant part; capturing an image of the relevant part; determining at least one treatment option for the treatment area based on the captured image; simulating a treatment outcome for the determined treatment option, producing a simulated image treated with the determined treatment option; and displaying recommendation for the determined treatment option with the simulated image and relevant information.

### CROSS-REFERENCE TO RELATED APPLICATIONS

This application claims priority to and the benefit of U.S. Provisional Patent Application No. 63/637,802, filed Apr. 23, 2024, the contents of all of which are hereby incorporated by reference herein for all purposes.

### TECHNICAL FIELD

Embodiments relate generally to product recommendations, and more particularly to cosmetic product recommendations.

### BACKGROUND

In recent years, the number of individuals seeking ways to enhance their facial and body aesthetics has increased. With advancements in technology and an increasing emphasis on self-care and personal grooming, more people are turning to medical treatments (surgical and non-surgical methods) to achieve their desired appearance.

The medical procedures may range from non-surgical procedures, such as Botulinum toxins injections, dermal fillers, Platelet-Rich Plasma (PRP) injections, laser treatments, to surgical interventions, such as rhinoplasty, facelifts, tummy tuck and liposuction. Individuals are increasingly willing to undergo these procedures to address perceived flaws or signs of aging and/or to achieve a more youthful look. These medical treatments offer immediate and dramatic results.

Meanwhile, while this pursuit of beauty is a personal choice, it is essential to approach both invasive and non-invasive medical treatments with informed decision-making. However, patients interested in these cosmetic procedures find it challenging or difficult to know what treatment options are available and how effective those treatments are for them. Since consultations with different doctors might provide different approaches, patients need to gather information and gain knowledge about the possible medical treatment options, risks, cost, and being able to find a provider. However, finding this information is not easy due to the lack of easily accessible resources tailored to their unique face and body features and preferences.

### SUMMARY

A system, device, and method is disclosed where a method embodiment may comprise: scanning, by a camera module in communication with an application installed on a user device, a relevant part of a face, head, or body of a user; mapping, by a computer vision module of the application, the scanned relevant part onto a mesh model to ensure consistent positioning, where the consistent positioning comprises whether the user device is being held upright using a combination of built-in motion and orientation sensors; positioning, by the computer vision module, the scanned relevant part in a correct position based on the mesh model, where the correct position comprises having a facial positioning in relation to the camera module that is at an angle that is less than a predetermined threshold; detecting, by a feature detection module of the application, a set of features associated with the scanned relevant part using the mesh model based on the scanned relevant part positioned in a correct position; determining, by an image classification module of the application, a treatment area that is needed to be treated based on the detected features associated with the scanned relevant part; capturing, by the camera module, an image of the relevant part based on the detected set of features; transmitting, by the camera module, the captured image to a treatment option determination module of the application; determining, by the treatment option determination module, at least one treatment option for the treatment area based on the transmitted captured image; simulating, by an image-to-image translation module of the application, a treatment outcome on the treatment area for each of the determined at least one treatment option, producing a simulated image of the treatment area treated with the determined at least one treatment option; and displaying, by a display module of the user device, recommendation for the determined at least one treatment option with relevant information, the relevant information including the simulated image and at least one of: the type of medical product, the volume and amount of the medical product, and the type of medical procedure.

The method may further comprise: determining, by the treatment option determination module, using machine learning neural networks, at least one treatment option for the treatment area based on the captured image.

In one embodiment, the detecting is based on performing a detailed analysis to label specific facial areas. In one embodiment, the detecting is based on performing a detailed analysis to label specific facial areas, where the specific facial areas comprise: lips, nose, and under-eye region. In one embodiment, the detecting is further based on identifying and classifying different parts of the relevant part for determining a set of appropriate treatment areas. In one embodiment, determining at least one treatment option is based on understanding characteristics and needs of each specific treatment area. In one embodiment, the understanding characteristics is based on recognizing signs of aging, dryness, or volume loss.

Another method embodiment for generating medical treatment recommendation may comprise: scanning, by a camera module of an application installed on a user device, a relevant part among the face, head, and body of a user; mapping, by a computer vision module of the application, the scanned relevant part onto a mesh model to ensure consistent positioning; positioning, by the computer vision module, the scanned relevant part in the right angle using the mesh model; detecting, by a feature detection module of the application, a set of features associated with the scanned relevant part using the mesh model; determining, by an image classification module of the application, a treatment area that is needed to be treated based on the detected features associated with the scanned relevant part; capturing, by the camera module, an image of the relevant part; transmitting, by the camera module, the captured image to a treatment option determination module of the application; determining, by the treatment option determination module, at least one treatment option for the treatment area based on the captured image; simulating, by an image-to-image translation module of the application, a treatment outcome on the treatment area for each of the determined at least one treatment option, producing a simulated image with the determined at least one treatment option; and displaying, by a display module of the user device, recommendation for the determined at least one treatment option with relevant information, the relevant information including the simulated image and at least one of: the type of medical product, the volume and amount of the medical product, and the type of medical procedure.

A disclosed system embodiment may comprise: a camera module in communication with an application installed on a user device; a computer vision module; a feature detection module; an image classification module; a treatment option determination module; an image-to-image translation module; and a display module; where the system may be configured to: scan, by the camera module, a relevant part of a face, head, or body of a user; map, by the computer vision module, the scanned relevant part onto a mesh model to ensure consistent positioning, where the consistent positioning comprises whether the user device is held upright via a combination of built-in motion and orientation sensors; position, by the computer vision module, the scanned relevant part in a correct position based on the mesh model, where the correct position comprises a facial positioning in relation to the camera module that is at an angle that is less than a predetermined threshold; detect, by the feature detection module, a set of features associated with the scanned relevant part using the mesh model based on the scanned relevant part positioned in a correct position; determine, by the image classification module, a treatment area that is needed to be treated based on the detected features associated with the scanned relevant part; capture, by the camera module, an image of the relevant part based on the detected set of features; transmit, by the camera module, the captured image to a treatment option determination module of the application; determine, by the treatment option determination module, at least one treatment option for the treatment area based on the transmitted captured image; simulate, by the image-to-image translation module of the application, a treatment outcome on the treatment area for each of the determined at least one treatment option, producing a simulated image of the treatment area treated with the determined at least one treatment option; and display, by the display module of the user device, recommendation for the determined at least one treatment option with relevant information, where the relevant information includes the simulated image and at least one of: the type of medical product, the volume and amount of the medical product, and the type of medical procedure.

The system may be further configured to: determine, by the treatment option determination module, using machine learning neural networks, at least one treatment option for the treatment area based on the captured image. In one embodiment, the system is configured to detect based on performing a detailed analysis to label specific facial areas. In one embodiment, the system is configured to detect based on performing a detailed analysis to label specific facial areas, where the specific facial areas comprise: lips, nose, and under-eye region. In one embodiment, the system is configured to detect further based on identifying and classifying different parts of the relevant part to determine a set of appropriate treatment areas. In one embodiment, the system is configured to determine at least one treatment option based on understanding characteristics and needs of each specific treatment area. In one embodiment, the system is configured to understand characteristics based on recognizing signs of aging, dryness, or volume loss.

### BRIEF DESCRIPTION OF THE DRAWINGS

The components in the figures are not necessarily to scale, emphasis instead being placed upon illustrating the principals of the invention. Like reference numerals designate corresponding parts throughout the different views. Embodiments are illustrated by way of example and not limitation in the figures of the accompanying drawings, in which:

FIG. 1A depicts a block diagram of a system for generating medical treatment recommendation, according to one embodiment;

FIG. 1B illustrates a schematic diagram of a system for generating medical treatment recommendation, according to one embodiment;

FIG. 2 depicts a flowchart of a method for generating medical treatment recommendation, according to one embodiment;

FIG. 3 depicts a step of mapping a scanned face part of a user onto a mesh model for a method for generating medical treatment recommendation, according to one embodiment;

FIG. 4 depicts a step of positioning a face part of a user in the right angle using the mesh model for a method for generating medical treatment recommendation, according to one embodiment;

FIG. 5 depicts steps of detecting facial features of a face part of a user and determining a treatment area of the face part, according to one embodiment;

FIG. 6 depicts a step of capturing an image of the face part for a method for generating medical treatment recommendation, according to one embodiment;

FIG. 7A depicts steps of determining at least one treatment option for a method for generating medical treatment recommendation, according to one embodiment;

FIG. 7B depicts a functional block diagram of the neural networks for treatment recommendations and outcome simulations, according to one embodiment;

FIG. 8 depicts steps of simulating a treatment outcome for a treatment option and producing simulated images, according to one embodiment;

FIG. 9 shows a high-level block diagram and process of a computing system for implementing an embodiment of the system and process;

FIG. 10 shows a block diagram and process of an exemplary system in which an embodiment may be implemented;

FIG. 11 depicts a cloud computing environment for implementing an embodiment of the system and process disclosed herein; and

FIG. 12 illustrates an example top-level functional block diagram of a computing device embodiment.

### DETAILED DESCRIPTION

The system and method embodiments of the present disclosure analyze each user's unique face and/or body structure to visualize potential treatment outcomes and recommend suitable medical treatments. That is, the results of visualization and recommendations that the system and method deliver are not arbitrary but grounded in the initial analysis of the facial and/or body structure. In addition, unlike an image editor, the results that the system and method deliver are aligned with actual medical treatments, taking into consideration each user's facial and/or body features. This ensures that the visualization and recommendations are not only technologically advanced but also practically applicable and tailored to each user's unique face and/or body characteristics. Furthermore, the visualization of the potential treatment outcomes that the system and method deliver is the actual image transformation executed through neural networks that are informed by real medical treatments, rather than relying on predefined editing criteria. Thus, the user is enabled to make a more informed decision based on their actual image showing the potential treatment outcomes.

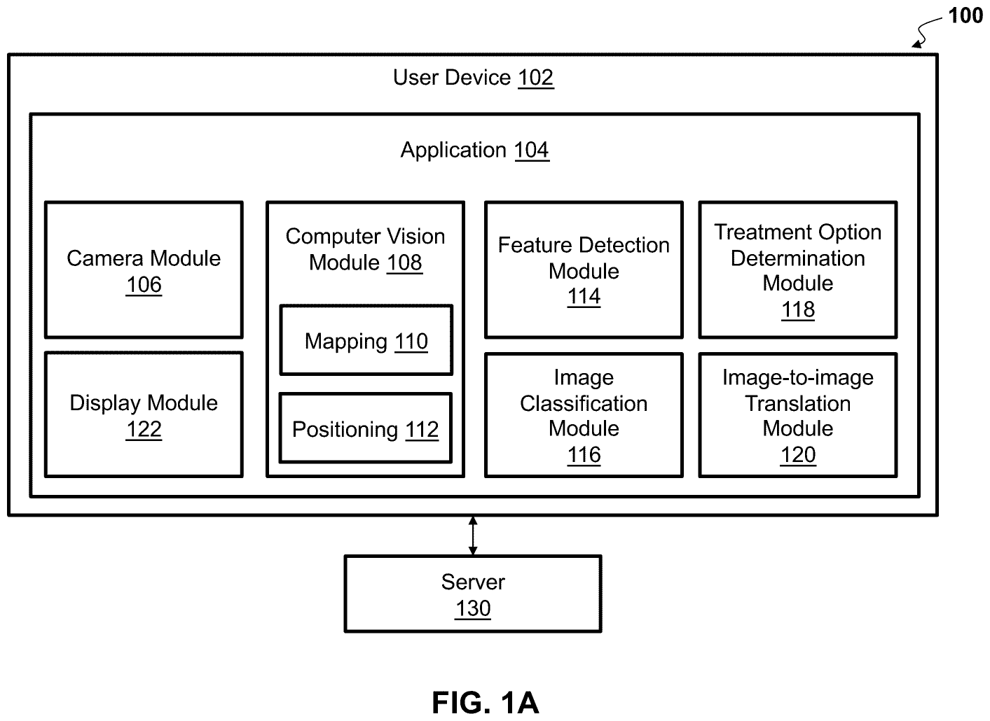

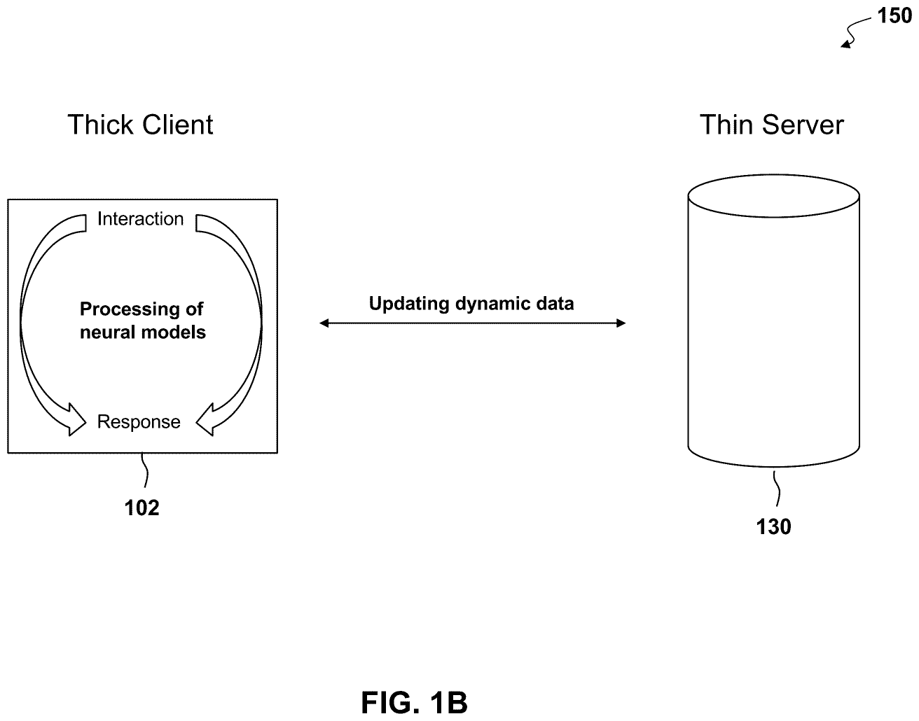

FIG. 1A depicts a block diagram of a system for generating medical treatment recommendation, according to one embodiment. Referring to FIG. 1A, the system 100 may comprise an application 104 that may be installed on a user device 102 and includes a camera module 106, a computer vision module 108 including a mapping configuration component 110 and a positioning configuration component 112, a feature detection module 114, an artificial intelligence (AI) driven image classification module 116, a treatment option determination module 118, an image-to-image translation module 120, and a display module 112. The application 102 may be provided and updated by a remote service provider server 130, but the system 100 does not need to be connected with the server 130 to generate medical treatment recommendations. The system 100 may locally execute the application 104 on the user device 102, such as smartphone, tablet PC, mobile device, and personal computing device, to generate medical treatment recommendations and provide them to the user. Accordingly, the system 100 enables the users to access virtual consultations for medical treatments while providing low-cost offline usage. Individuals who are interested in and/or seek information on medical treatments to enhance their appearance may obtain knowledge of medical treatment options without visiting doctors or paying consultation fees. In addition, the service provider for the system 100 may also implement a fat client-thin server architecture. That is, in one embodiment a fat client (also called a thick client) is used to perform the bulk of the processing in client with a reduced reliance on server applications. Whereas a thin client network computer, without a hard disk drive, acts as a simple terminal to the server, requiring constant communication with the server.

FIG. 1B illustrates a schematic diagram of a system for generating medical treatment recommendation, according to one embodiment. Referring to. FIGS. 1A and 1B, the service provider for the system 150 may implement a fat/thick client-thin server architecture. The system 150 may be configured to locally operate the application 104 on the user device 102 (client) without connecting to the service provider server 130. The service provider server 130 may communicate with the client 102 for updating dynamic data but does not need to be connected with the user device 102 while generating the cosmetic medical treatment recommendation on the user device 102. Thus, the service provider server 130 does not need to have the huge space for a plurality of clients 102 to operate the system 150, and each client 102 may use the system 150 without any issues caused by other clients.

In the offline environment of the user device 102, the user may interact and receive response through the processing of neural models or neural networks provided by the system 150. Accordingly, the system 150 may be configured to enable the user to access virtual recommendations and consultations for cosmetic medical treatments with low-cost offline usage.

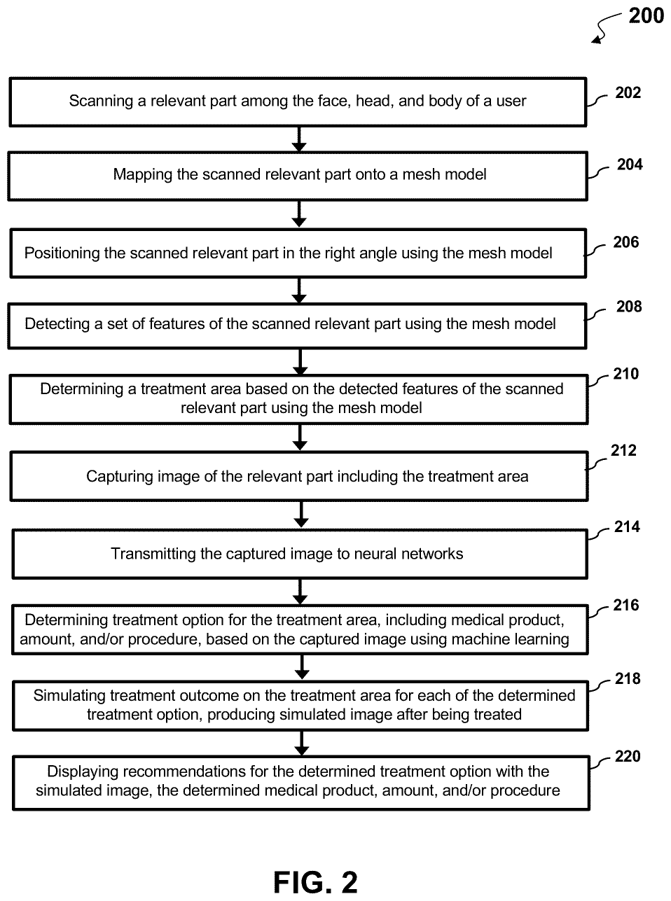

FIG. 2 depicts a flowchart of a method for generating medical treatment recommendation, according to one embodiment. Referring to FIGS. 1A, 1B, and 2, the method 200 for generating medical treatment recommendation may comprise: scanning, by a camera module 106 of an application 104 installed on the user device 102, a relevant part among the face, head, and body of a user (step 202); mapping, by a computer vision module 108 of the application 104, the scanned relevant part onto a mesh model to ensure consistent positioning (step 204); positioning, by the computer vision module 106, the scanned relevant part in the right angle using the mesh model (step 206); detecting and/or determining, by a feature detection module 114 of the application 104, a set of features associated with the scanned relevant part using the mesh model (step 208); analyzing, identifying, and determining, by an image classification module 116 of the application 104 using neural networks, a specific treatment area that is needed to be treated based on the detected features associated with the scanned relevant part using the mesh model (step 210); capturing, by the camera module 106, an image of the relevant part of the treatment area, for example, the face, head, and/or body (step 212);transmitting, by the camera module 106, the captured image to a treatment option determination module 118 of the application 104 (step 214); determining, by the treatment option determination module 118 using machine learning neural networks, at least one treatment option for the treatment area based on the captured image (step 216); simulating, by an image-to-image translation module 120 of the application 104, a treatment outcome on the treatment area for each of the determined at least one treatment option using neural networks, producing a simulated image treated with the determined at least one treatment option (step 218); and displaying, by a display module 122 of the user device 102, recommendation and advice for the determined at least one treatment option with relevant information (step 220).

Specifically, individuals who are interested in and/or seek information on medical treatment to improve their appearance may install the application 104 of the system 100 on their device 102. Thus, the user of the system 100 may be patients or potential clients of medical treatments. Using the camera module 106 of the application 104, any relevant part among the face, head, and body of the user may be scanned (step 202). In this case, the scanned relevant part may be mapped onto a mesh model by a computer vision module 108 to ensure consistent positioning (step 204) where the consistent positioning may be to ensure the device being used to take the image is being held upright.

In one embodiment, the step of “Scanning” may refer to the real-time process where the user device, e.g., smartphone, detects and analyzes a user's face using the camera, for example, front-facing camera, or dedicated sensors. The step of “Capturing” may be the discrete moment when the system records a facial image or facial features for the disclosed purposes. In one of the disclosed embodiments, a set of functions may be executed where the functions may include: Face detection using computer vision; Pose estimation (e.g., checking face angle, lighting, occlusion); Liveness detection (e.g., blinking, movement) to prevent spoofing; and Depth scanning. In addition, the “Mapping” and “Positioning” steps may include the user device determining whether it is being held upright (e.g., in portrait orientation and not lying flat or upside-down) using a combination of built-in motion and orientation sensors, particularly: Accelerometer which may measure linear acceleration along the X, Y, and Z axes; Gyroscope which may measure rotational velocity around the three axes to help track how the device is being rotated in real time (pitch, roll, yaw); Magnetometer (Digital Compass) which may measure the ambient magnetic field to determine the phone's orientation relative to Earth's magnetic north and help determine the absolute orientation—often combined with other sensors; and a set of Sensor Fusion Algorithms executed on the device's operating system which may combine data from the accelerometer, gyroscope, and magnetometer to calculate a more accurate and stable orientation. Using the above, the application 104 may be configured to determine whether the phone is being held vertically or horizontally and optionally, whether the phone is facing the user or away (important for facial scanning and capturing of the image).

The orientation of the user device may be particular needed in the presently disclosed system due to the effect of Gravity on the body part (especially the face) which pulls on the skin and body parts that may introduce errors or unwanted results. That is, by detecting which axis experiences gravity, the phone can infer its orientation and thereby detect whether the user is in the right position (upright and not laying down) while going through this process. Accordingly, the method for generating medical treatment recommendation may also include the steps of: sensing, by the accelerometer, gravity on the Y-axis, indicating that the device is vertical; confirming, by the gyroscope, that the device isn't rotating erratically, indicating that the device is stable; refining, by the magnetometer, the phone's orientation/direction based on whether the phone is facing north/up or not; and classifying the orientation as portrait upright to adjust the mesh model or enable notification feature to inform the user of the adjustment needed.

The above steps may be executed via an Inertial Measurement Unit (IMU) of the user device which is typically hardware (a chip containing accelerometer+gyroscope±magnetometer). The steps may also be executed via an Attitude and Motion Unit (AMU), which may be a software/hardware fusion system that combines data from inertial sensors to determine the device's orientation, motion, and position in 3D space. An AMU may be the software stack (or software+firmware) that processes IMU data using filtering algorithms to produce a usable, real-time estimate of the device's motion and orientation.

In addition to the error detection attributed to the positioning of the user device, as disclosed herein, the system and method may include embodiments for creating a mesh model and drawing a vector from the nose of the user to the camera to make sure the vector is, for example, at a 90 degree angle to the camera based on a threshold tolerance (e.g., +/−5 degrees) and therefore in the correct angle or correct position. If the vector is outside the predetermined threshold tolerance, then the system may be configured to not allow the user to take the photo/image.

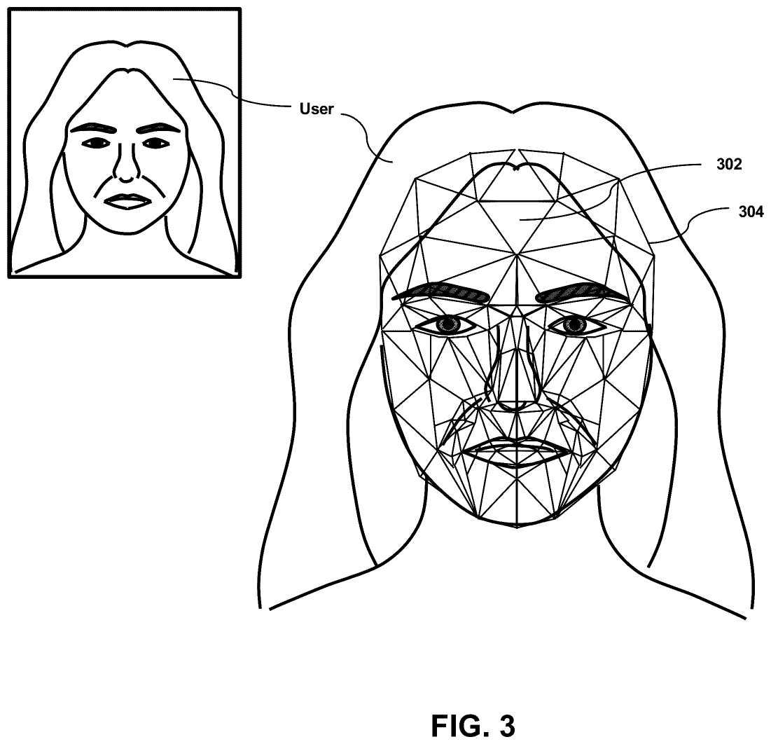

FIG. 3 depicts a step of mapping a scanned face part of a user onto a mesh model for a method for generating medical treatment recommendation, according to one embodiment. Referring to FIGS. 1A-3, the user interested in medical treatments for their face may scan their face (or parts thereof) 302 using the camera module 106 (step 202). Once the camera module 106 scans their face part 302, the computer vision module 108 may collect point cloud data from the scanned face part 302 in the background, which is essential for creating a detailed mesh model, and map the point cloud data of the scanned face part 302 onto a mesh model 304. The point cloud data may be a set of data points in a three-dimensional coordinate system forming a 3D representation of an object or environment. Each cloud point may represent a single spatial measurement on the surface of the object, such as the face part 302. The mesh model 304 may form a structural build of three-dimensional model consisting of polygon for the face part 302 as a digital representation of the face, which may be constructed in the background. In some embodiments, this mesh model may not be displayed to the user. The mesh model 304 derived from the point cloud data may provide insights into x-y positioning of the face within the frame of the camera module 106 and help the system 100 understand the face's orientation, angle, and distance from the camera module 106.

The mesh model 304 of the present disclosure may play a crucial role in the accurate analysis and improvement process, especially in the spatial orientation, facial proportions analysis, and depth and volume assessment. By collecting the point cloud data and creating the mesh model 304 in the background, the system 100 may conduct a comprehensive and detailed analysis of the face part 302. This process of the system 100 underpins the ability to provide customized recommendations for cosmetic improvements using the medical treatments, based on the unique facial structure and aesthetic goals of the user.

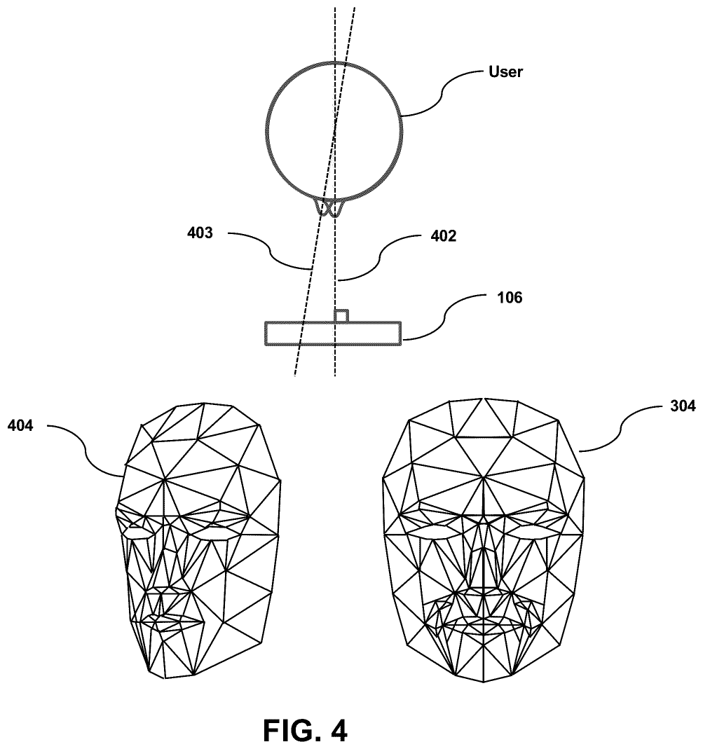

Specifically, the mesh model 304 of the face part 302 may be used in positioning the face part 302 in the right angle (step 206). FIG. 4 depicts a step of positioning a face part of a user in the right angle using the mesh model for a method for generating medical treatment recommendation, according to one embodiment. Referring to FIGS. 1A, 2, and 4, the positioning configuration component 112 of the computer vision module 108 may help the user position their face part (302, FIG. 3) for example, in front of the camera module 106. In incorrect orientations 403 where the nose of the user does not face the camera module 106 at a 90-degree angle, the user may recognize the incorrect face orientation from a mesh model 404 formed in an asymmetrical polygon layout and/or notifications from the device 102 indicating as such. The system 100 may also inform the user to correct the face orientation. In a correct orientation 402 where the nose of the user faces the camera module 106 at a 90-degree angle, the user may recognize the correct face orientation from a mesh model 304 formed in a symmetrical polygon layout. In one embodiment, the system 100 may also inform the user that the correct orientation is detected. This way, the mesh model 304, 404 may be used to ensure consistent facial positioning.

The mapping configuration component 110 may use artificial intelligence (AI) technology. The AI technology tapping into deep learning algorithms may be used to implement highly accurate facial mapping for a true-to-life makeover experience, using current medical knowledge. In some embodiments, the steps of scanning (step 202), mapping (step 204), and the positioning (step 206) may use the camera module 106, augmented reality (AR) module, light detection and ranging (Lidar) module, and/or face detection/recognition module. The camera module 106 may scan the face part, and the face detection/recognition module may help the user position the face part in the right angle. These configurations allow the camera module 106 to take consistent face scans or images and enable the system 100 to analyze the face part properly.

The application 104 may optionally further comprise the Lidar module. The Lidar module may utilize a mesh model of the face to accurately position it within the view of the camera module 106 and identify the specific facial features, such as the eyes. Then, the Lidar module may create a detailed 3D model of the face based on the identified facial features. In some embodiments, a depth analyzing module, such as TrueDepth function, may be employed instead of the Lidar module. The depth analyzing module may capture precise facial data by projecting and analyzing invisible dots to create a depth map. The application 104 may further comprise the AR module. The AR module may be employed to overlay a virtual face mask on the user's face in real-time, aiding in the visualization of potential aesthetic enhancements. Additionally, the AR module may be used to pinpoint the exact locations for medical treatments, such as Botulinum toxin treatments, which are then projected onto a 2D image to provide the user with a visual representation of the treatment outcome.

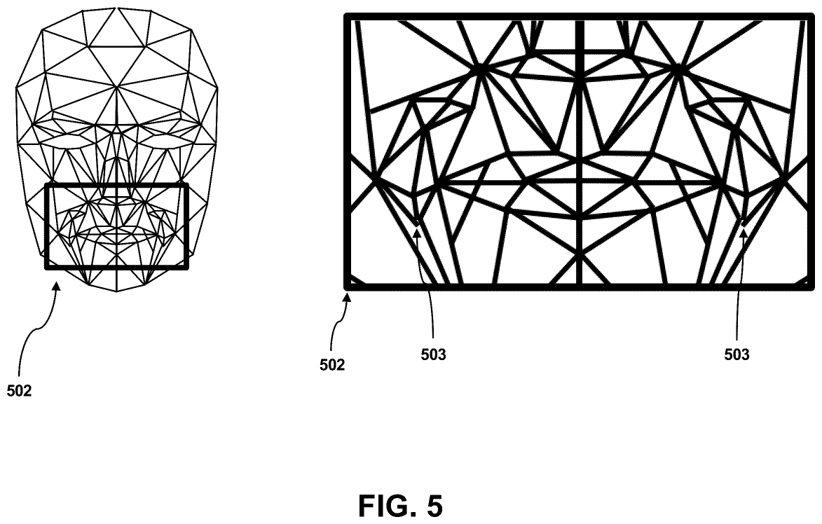

In addition to ensuring consistent positioning, the mesh model 304 mapped with the face part may also be used for facial analysis. Specifically, the mesh model 304 may be used to determine the facial features by the feature detection module 114 (step 208) and identify specific treatment areas by the image classification module 116 (step 210). FIG. 5 depicts steps of determining facial features of the face part and determining a treatment area 502 of the face part, according to one embodiment. Referring to FIGS. 1A, 2, and 5, the mesh model 304 derived from the point cloud data may provide information of x-y positioning, orientation, angle, and distance of a face from the camera module 106 and allow the feature detection module 114 to accurately interpret facial features and analyze facial proportions under various conditions. This analysis may include examining the ratios and balance among the facial features, such as the eyes, nose, and mouth, contributing to the perceived beauty and symmetry of the face.

The mesh model 304 may also enable the feature detection module 114 to assess the facial depth and volume in detail and allow the image classification module 116 to identify and determine areas that may appear hollow or require volume enhancement. That is, the image classification module 116 may detect and determine the specific treatment area 502 that is needed to be treated based on the facial features determined by the feature detection module 114. For example, if there is volume loss in the face, the image classification module 116 may recognize the area that is not proportional and may determine this as a treatment area 502. In the example shown in FIG. 5, the image classification module 116 may detect nasolabial folds 503, which are not proportional or smooth, using the mesh model 502 and determine this as the treatment area 502. This treatment area 502 may be used for facial analysis, improvement, and highlighting to explain medical treatments. In some embodiments, the step of determining the treatment area (step 210) may include determining a volume loss and a depth of the volume loss of the treatment area to be corrected.

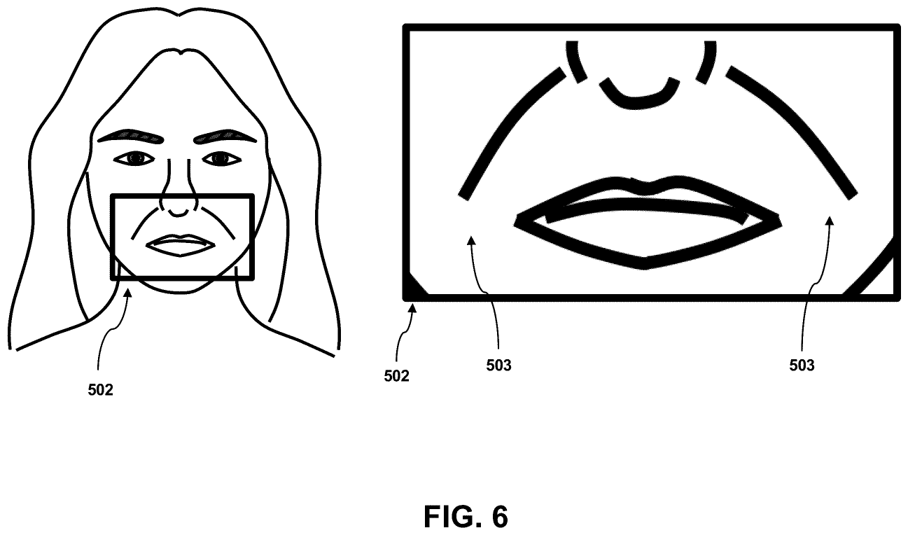

Once the treatment area 502 is determined, the camera module 106 may capture images of the face part including the treatment area 502 (step 212). FIG. 6 depicts a step of capturing an image 605 for a method for generating medical treatment recommendation, according to one embodiment. Referring to FIGS. 1A, 2, and 6, the captured image 605 may include a mesh model. In some embodiments, the captured image 605 may also be one that does not include a mesh model. Then, the captured image 605 may be fed to the treatment option determination module 118 of the application (step 214). By utilizing the captured image 605, the system 100 may feed neural networks to recommend treatments.

Based on this analysis for the features and treatment area 502, the treatment option determination module 118 may make qualitative recommendations regarding the types and quantities of cosmetic medical treatments needed, ensuring a tailored approach to facial enhancement. Specifically, the treatment option determination module 118 may determine at least one treatment option based on the captured image 605 including the treatment area 502 (step 216).

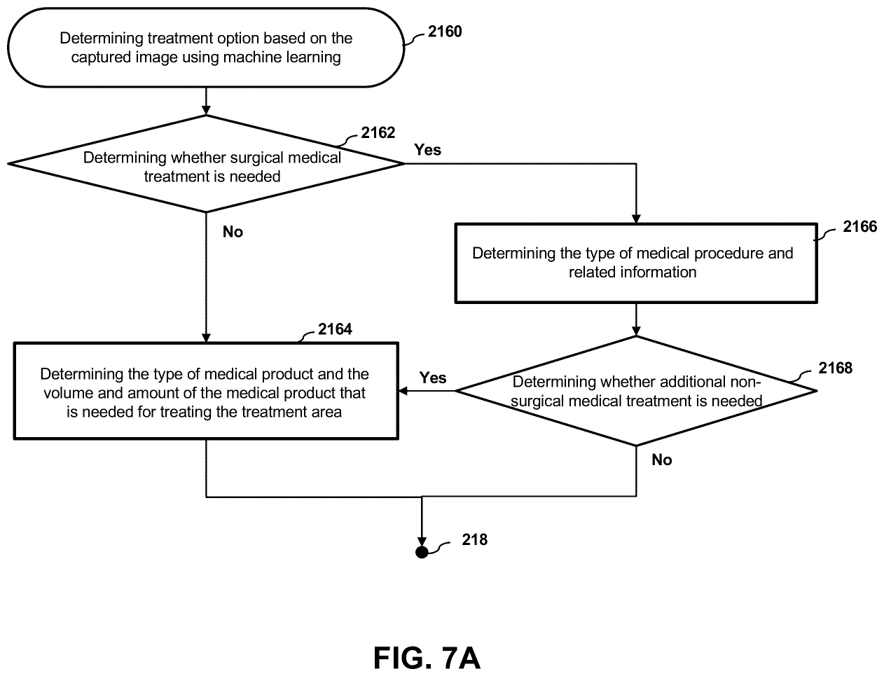

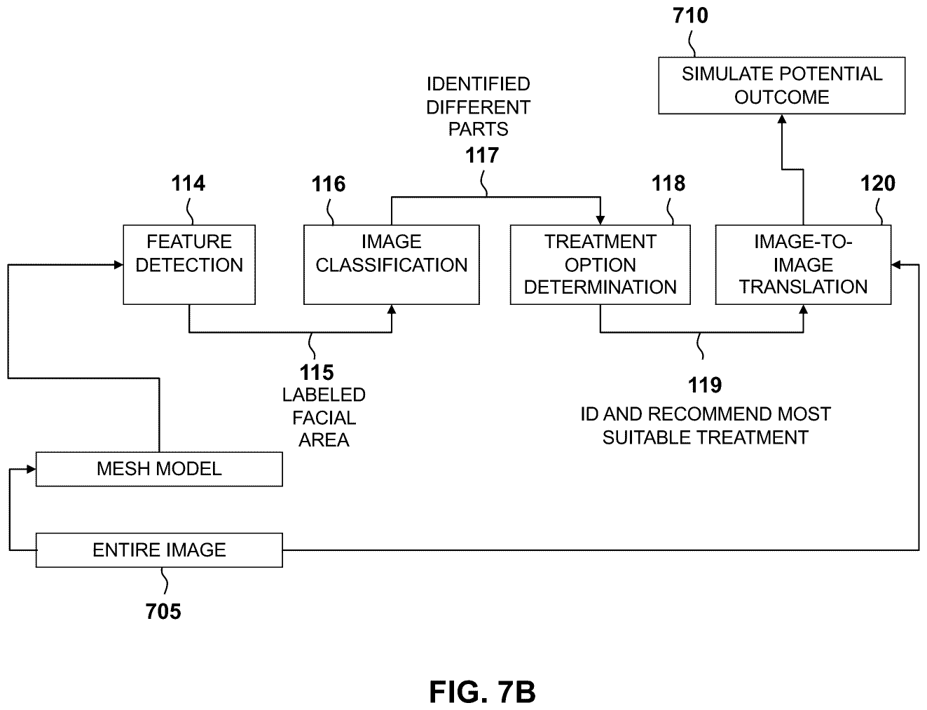

FIG. 7A depicts steps of determining at least one treatment option, according to one embodiment. Referring to FIGS. 1A, 2, and 7, the step of determining at least one treatment option based on the captured image using, for example, machine learning (step 2160) and the steps may include: determining whether a surgical medical treatment or a non-surgical medical treatment is needed (step 2162); determining, based on when the surgical medical treatment is not needed, at least one type of medical product and the volume and amount of the medical product that is needed for treating the treatment area (step 2164); determining, based on when the surgical medical treatment is needed, at least one of type of medical procedure (step 2166); determining whether additional non-surgical medical treatment is needed as well as the surgical medical treatment (step 2168); and outputting a simulation of a treatment outcome for each of the determined at least one treatment options, producing a simulated image of the treatment area treated with the determined at least one treatment option (step 218).

In some embodiments, the step of determining the at least one of the type of medical product and the volume and/or amount of the medical product may be performed based on the treatment area 502, including the volume loss and the depth of volume loss, the density, consistency, and G′ prime (G′) of the medical product that should be injected in the treatment area 502. The density, consistency, and G′ prime (G′) may refer to the physical properties of the medical product, such as a dermal filler. As different materials have different density, consistency, and elasticity, various dermal fillers also have different physical properties. The consistency relates to the dermal filler's thickness or fluidity, impacting how it spreads and settles in the tissue. The G′ prime indicates the dermal filler's elasticity and ability to maintain shape under stress. That is, in addition to the recommendations of a suitable filler and the volume and/or amount of it, the treatment option determination module 118 of the application 104 may determine the ideal filler consistency and G′ prime for achieving desired aesthetic results based on evaluation or determination of the facial features and the treatment area. Accordingly, the treatment option determination module 118 may determine what type of medical product is needed and then calculate how much of the medical product is needed and/or what quantity of the respective product to use is needed in the given treatment area for volume loss and the area being treated to achieve optimal and natural appearing results, ensuring precise, tailored treatment plans. Therefore, the application 104 may be configured to determine the types of filler with the density, consistency, and G′ prime, which are needed in certain parts of the face and body.

The treatment option determination module 118 may use machine learning to determine the treatment option. Thus, the system 100 may then make recommendations similar to a practitioner by training the treatment option determination module 118 with the expertise of medical experts for their areas of expertise. As described above, the treatment option may include surgical medical treatment and non-surgical medical treatment, such as Botulinum toxin, fillers, lasers, light therapy, and others.

Once at least one treatment option is determined, the image-to-image translation module 120 may simulate a treatment outcome for each of the determined at least one treatment option and produce a simulated image of the treatment area treated with the determined at least one treatment option (step 218).

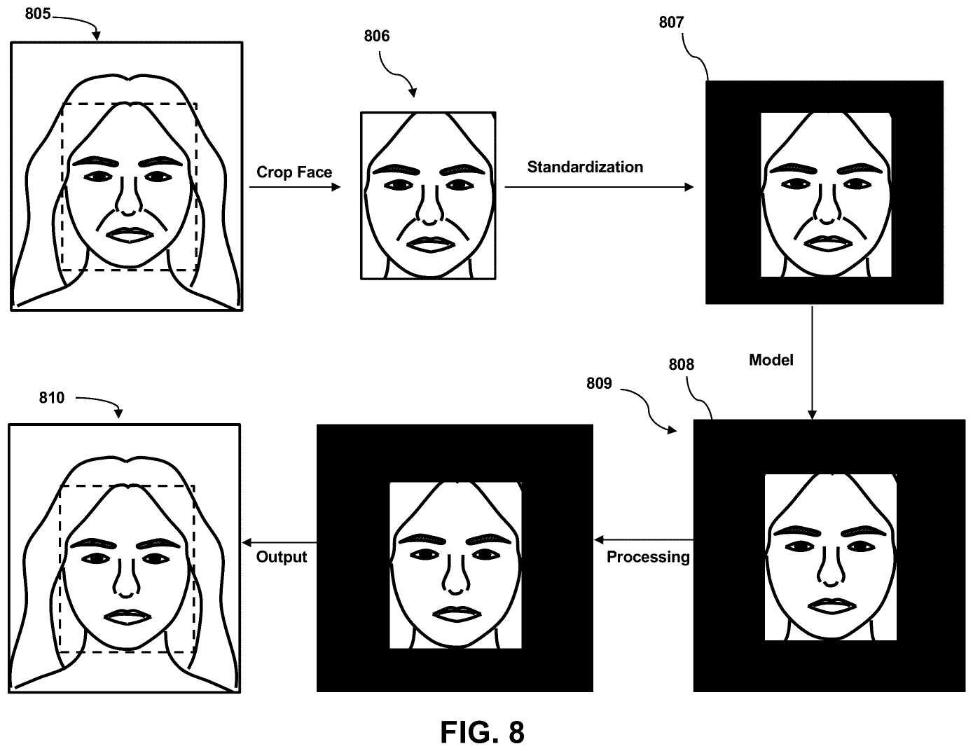

FIG. 7B depicts a functional block diagram of the neural networks for treatment recommendations and outcome simulations, according to one embodiment. In the disclosed system the entire image 705 may be received from the camera module where it is stored and also a mesh model may be generated from the image as disclosed herein. The generated mesh model may be transmitted as an input to the feature detection module 114 where the feature detection module 114 may be configured to detect facial features on the mesh model and label the facial areas to then transmit the labeled facial area 115 to the image classification module 116. In one example, the labeled facial areas, may be such as the lips, nose, and under-eye region. The image classification module 116 may then classify different parts of the face and transmit the identified different parts 117, which may also include the labeled areas, to the treatment option determination module 118 as input. The treatment option determination module 118 may be configured to identify and suggest the most suitable cosmetic product or medical treatment option for each area based on recognized signs of aging, dryness, or volume loss. The treatment option determination module 118 may identify and recommend the most suitable treatment 119, for example, a hydrating filler for the nasolabial folds or the under-eye area, or a volumizing product for the lips. In this embodiment, the image-to-image translation module 120 may receive as input the entire image 705 from the camera module along with the identified and recommended most suitable treatment 119. The image-to-image translation module 120 may then crop out the isolated image (see FIG. 8, ref. no. 806) from the entire image 805 of the face and apply transformations on the isolated image based on the recommended treatment/products. At this point, the simulate potential outcome 710 with the treatments applied, as a transformed image that shows how the face might look after the treatment.

FIG. 8 depicts steps of simulating a treatment outcome when the user uses the determined treatment option, according to one embodiment. Referring to FIGS. 1A and 8, the image-to-image translation module 120 may learn the mappings from the input images with facial imperfections to the output images with enhanced features (e.g., removing nasolabial folds, eye bags, and enhancing lips) and vice versa and then may transform an input image 805 with facial imperfections (e.g., nasolabial folds) to an output image 810 with enhanced features (e.g., removing nasolabial folds) based on the mappings. The image-to-image translation module 120 may ensure that the transformations are bijective where every element in the input image to exactly one element in the output image, and every element in the output image has exactly one element mapping to it from the input image, and thus the transformed image may always revert back to the original image if needed.

In one of the disclosed embodiments, bijective functions offer advantages primarily through their ability to be inverses and provide one-to-one mappings. That is, each input uniquely corresponds to an output, and each output uniquely corresponds to an input, enabling efficient data transformations, retrieval, and storage. In one embodiment, by ensuring a one-to-one mapping, bijective functions may optimize data storage and retrieval. For example, in databases, a bijective function may map a unique key (like an ID) to a specific data record, allowing for fast access and retrieval. This may be akin to a databinding function to allow, for example, data compression algorithms to represent data in a more compact form, while maintaining the ability to reconstruct the original data. Bijective functions may be used in the disclosed system since in one use case, the images of the patients may be needed to be protected as part of the Health Insurance Portability and Accountability Act (HIPAA) compliance and privacy. That is, in one embodiment, cryptographic algorithms may be executed by the system where data needs to be uniquely transformed (encrypted) and then uniquely transformed back (decrypted). The unique mapping ensures that the original data may be recovered from the encrypted version. Additionally, in some embodiments, to ensure HIPAA compliance, the system may execute any steps related to the user's image or information locally on the user device. That is, the models may be downloaded and executed locally on the image being stored on the user device, thereby eliminating any user-server (client-server) communication of protected data. In another embodiment, an edge computing model may be implemented where a distributed computing paradigm in which data processing, storage, and computation occurs at or near the physical location where the data is generated rather than relying on a centralized cloud data center. This minimizes latency, reduces bandwidth usage, and enables real-time responsiveness by executing workloads on local devices such as IoT nodes, gateways, routers, smartphones, or micro data centers.

In one embodiment, facial recognition technology and image classification may be utilized to analyze a person's facial features to make recommendations for medical cosmetic (Botulinum toxin, fillers, lasers, surgical and non-surgical) improvements. The disclosed image-to-image and image classification neural networks may be converted into formats that are configured to run on a user device, such as a smart phone or other mobile devices without the need of remote server based processing via enhancing the image based on medical recommendations from local processing thereby achieving low-cost offline usage and independent usage (see fat client-thin server architecture discussed previously).

Additionally, image classification may be used to determine the treatment areas, including volume loss, depth of treatment area, and how many/much product is needed to improve the area. AI (via machine learning and training to teach a model to recognize patterns and make predictions by feeding it data, allowing it to learn and improve its performance over time) may be configured to determine what products to apply and what quantity of the respective product to use to achieve optimal and natural appearing results. The system may then make recommendations similar to a practitioner by training it with the expertise of medical experts for their areas of expertise.

The various embodiments disclose a system that may be configured to achieve lossless image to image translation. That is, all images are placed on a big, predefined frame and fill the blank space with black pixels. The input image and output image utilize the size of the frame that is much larger than the image the system is configured to translate itself. The model may be trained to always translate the black pixels to black pixels, therefore they will not impact the final outcome. However, the respective images placed on the frame remain their original size and do not require any rescaling. After placing the image on a bigger scaled frame, the model crops the same area of the original image again.

Specifically, the image-to-image translation module 120 may take the entire image 805 from the camera module 106. The neural network of the image-to-image translation module 120 may be fed with the entire image 805 by the camera module 106. Then, the image-to-image translation module 120 may crop out an isolated image 806 corresponding to the face from the entire image 805 to apply transformations on the isolated image 806 based on the recommended products. For standardization process, the image-to-image translation module 120 may need a pre-defined input image frame 807 and output image frame 808. The isolated image 806 may be put on pre-defined input image frame 807 that may be bigger than the isolated image 806, and the remaining blank space in the input image frame 807 may be filled with black pixels. The input image 805 and output image 810 may utilize the sizes of the input and output image frame 807, 808 that are much larger than the input image 805 and output image 810 the user actually wants to translate itself. In this embodiment, during the model process, the image-to-image translation module 120 may then simulate the potential outcomes of the treatment options on the face of the isolated image 806, put on the input image frame 807, and produce a transformed image 809 that shows how the face might look after the treatment option. The transformed image 809 may correspond to the input image frame 807 and may include a transformed isolated image with enhanced features on the output image frame 808. The model process may refer to the application of a Generative Adversarial Network (GAN) that learns to convert images from one domain to another without paired examples. The tasks of the image-to-image translation module 120, such as modifying facial features, for example, removing the Nasolabial fold, may be performed through this model process. The image-to-image translation module 120 may be trained to always translate the black pixels to black pixels, therefore they will not impact the final outcome.

The isolated image 806 put on the input image frame 807 and the transformed isolated image of the transformed image 809 may remain their original size and not require any rescaling. After the model process, in one embodiment, the image-to-image translation module 120 may perform further post-processing to refine the output on the transformed image 809 from the model process. This may include enhancing image quality, correcting any distortions or anomalies introduced during the transformation, and ensuring that the modifications, such as the removal of nasolabial folds, appear natural and consistent with the rest of the image. Then, the image-to-image translation module 120 may crop the same area of the original isolated image 806 again from the transformed image 809. This transformed isolated image cropped from the transformed image 809 may then be superimposed back onto the original entire image 805, providing the output image 810, which in this embodiment is a comprehensive visual representation of the treatment effects. This way, the image-to-image translation module 120 may achieve lossless image-to-image translation.

The image-to-image translation module 120 may use AI technology and neural networks. The AI technology with deep learning algorithms of the system 100 may reshape, remap, and contour the facial features based on the determined treatment option, including medical suggestions, medical advice, and consultations, and provide knowledge and/or opportunities to the user to determine the certain medical treatment options are effective enough to achieve their desired appearance and youthfulness.

The image-to-image translation module 120 and the image classification module 116 of the present disclosure may convert images into formats and configured to be processed on the user device, such as smartphone, tablet PC, or other mobile devices. Thus, the system 100 may locally operate to generate medical treatment recommendations without the need of the remote service provider server 130 based processing, such as processing through a set of remote servers, also called “cloud”. In one embodiment, the processing may be executed on the remote server side or alternatively, split processing between the client and server side.

While many sophisticated computer programs exist for digital photograph editing, the present disclosure provides an easy-to-use virtual medical treatment and/or makeover systems 100, 150, and methods 200 that produce a lifelike personalized rendering of an individual using medical cosmetic products and/or procedures, such as botulinum toxin, filles, lasers, light therapy, surgical treatments, and others. The term “lifelike” may mean “resembling or simulating real life; appearing as in a digital image that has not been retouched or graphically modified; a digital rendition of human face that appears to be of photographic quality; a high fidelity image; realistic in appearance.” The term “photorealistic rendering” or “photorealistic” may be used in the general sense of its meaning in computer graphics, such as “rendering images so that they closely resemble a photograph; such renderings take into account reflective properties, light sources, illumination, shadows, transparency and mutual illumination.” However, the term photorealistic may also be used in the sense that it is well understood by those of ordinary skill in the art of computer graphics but is not limited to that definition.

Once the treatment options are determined and simulated images are produced, the display module 122 may display to the user the recommendation and advice for the determined at least one treatment option with relevant information including simulated images showing the treatment result (step 220). In this case, in addition to the simulated output images 810, the other relevant information may include the type of medical product, the volume and amount of the medical product, and/or the type of medical procedure. In some embodiments, the relevant information may further include medical product details and prices. In some embodiments, the relevant information may further include at least one doctor who offers the determined treatment option.

As described above, the systems 100, 150, and method 200 of the present disclosure may help patients determine treatment areas needed to treat and make them appear more youthful and younger along with the treatment options, including how much of certain products are needed to achieve those results. In addition, the system 100 and method 200 may be configured to determine whether surgical or non-surgical medical treatments are needed based on the facial and/or body features of clients or patients through machine learning (ML) of artificial intelligence (AI). The system 100 and method 200 may also determine and/or recommend the type of product to be used based on its density and consistency as well as how much of the product is needed to be applied in the given area for volume loss and the area being treated.

Additionally, the systems 100, 150, and method 200 may depict realistic outcomes based on the application of these medical interventions. That is, the systems 100, 150, and method 200 may generate the image of facial reshape of the patient to allow them to see the results of certain treatment. The use of neural networks for treatment recommendations and outcome simulations is not only enhancing treatment efficacy but also contributing to higher levels of patient satisfaction. This iterative approach to treatment refinement based on real-world data ensures continuous improvement in treatment protocols, ultimately benefiting both patients and medical treatment providers.

Accordingly, the systems 100, 150, and method 200 may successfully enable users to access virtual free consultations for medical treatments. This platform offers users knowledge of medical treatment options to improve their appearance to be more youthful and younger and may conveniently view product details, prices, and information about various treatments, allowing them to select specific treatments based on their preferences.

Furthermore, the systems 100, 150, and method 200 facilitate the discovery of doctors who offer these treatments, enhancing the overall user experience and accessibility to healthcare services. The systems 100, 150, and method 200 also lead to an increase in knowledge of individuals opting for medical cosmetic treatments, driven by greater confidence in understanding potential outcomes, treatment plan, and facial and body analysis in advance. An individual may understand the potential or capabilities of the medical procedures (surgical or non-surgical) without visiting a medical professional by using the disclosed application 104 of the present disclosure. The application 104 may be configured to recommend the best method to achieve youthful appearance prior to making any commitments. The application 104 may be configured to determine whether one will need surgical intervention or non-surgical procedure to achieve the best results. For example, as mentioned above, the evaluation of the face may allow the user to know if any non-surgical method for lifting the face can be used or only surgical treatment would be the solution. This information provided by the application 104 results in fewer instances of regretful procedures, as patients can visualize and anticipate their post-treatment appearance more accurately with methods that might be more easily achieved than previously thought. Moreover, patients now come to appointments better prepared, armed with a clearer understanding of their desired outcomes, and having identified the specific areas they wish to address. This preparedness streamlines the consultation process, fostering more meaningful discussions between patients and medical cosmetic treatment providers and leading to more personalized treatment plans.

Therefore, the systems 100, 150, and method 200 disclosed herein may enhance the understanding of cosmetic medical products and/or procedures and improve the accessibility to resources for individuals seeking medical treatments including surgical and non-surgical treatments.

The disclosed solutions of the present disclosure offer several advantages over the prior solutions. Firstly, it is specifically designed for end users, ensuring ease of use, friendliness, and cost-efficiency. Additionally, the systems 100, 150, and method 200 provide free consultations for medical treatments, which were not available in the prior solution. Furthermore, users may now access comprehensive information about treatments and treatment options, including product details and prices, leading to a more informed decision-making process.

In the systems 100, 150, the neural networks play a crucial role in analyzing facial images and recommending treatment options. The process of the systems 100, 150 may involve several steps, each utilizing different aspects of neural networks. In the initial analysis with the feature detection module 114 and the image classification module 116, the first neural network model may take the mesh model of the entire image as input and perform a detailed analysis to label specific facial areas, such as the lips, nose, and under-eye region. This step may involve identifying and classifying different parts of the face, which are crucial for determining the appropriate treatment areas.

In the second analysis with the treatment option determination module 118, once the facial areas are labeled, another neural network model may take these labeled areas as input. This model may specialize in understanding the characteristics and needs of each specific area, such as recognizing signs of aging, dryness, or volume loss. Based on this detailed analysis, the treatment option determination module 118 may identify and recommend the most suitable cosmetic product or medical treatment option for each area. For example, the treatment option determination module 118 might suggest a hydrating filler for the nasolabial folds or the under-eye area, or a volumizing product for the lips.

In the third analysis with the image-to-image translation module 120, a third neural network of the image-to-image translation module 120 may take the entire image 805 again from the camera module 106. The third neural network of the image-to-image translation module 120 may be fed with the entire image 805 from the camera module 106. Then the image-to-image translation module 120 may crop out the isolated image 806 from the entire image 805 of the face and apply transformations on the isolated image 806 based on the recommended products. This step may simulate the potential outcomes of the treatments on the face, producing a transformed image 810 that shows how the face might look after the treatment. This transformed isolated image cropped from the transformed image 809 may then be superimposed back onto the original entire image 805, providing the output image 810, which shows a comprehensive visual representation of the treatment effects.

In summary, the systems 100, 150 may be configured to use a combination of the image classification module 116 for analyzing the face, the treatment option determination module 118 for recommending treatment options, and the image-to- image translation module 120 for visualizing the potential results. This multi-step neural network process ensures a detailed and accurate recommendation and visualization of cosmetic medical treatments, enhancing the decision-making process for both the provider and the user.

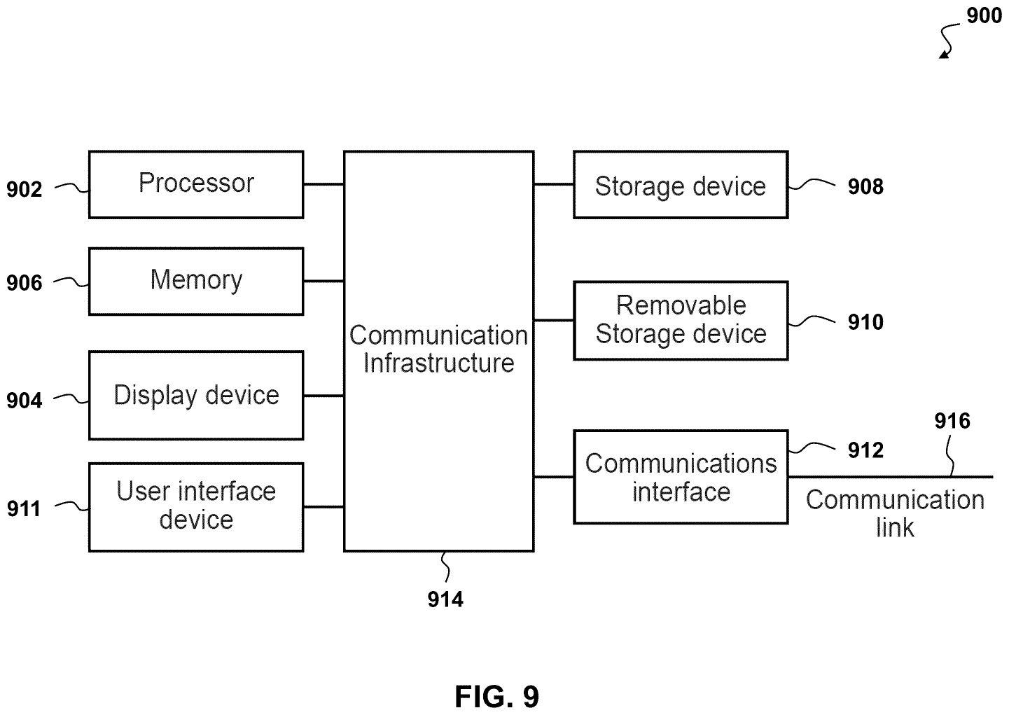

FIG. 9 is a high-level block diagram 900 showing a computing system comprising a computer system useful for implementing an embodiment of the system and process, disclosed herein. Embodiments of the system may be implemented in different computing environments. The computer system includes one or more processors 902, and can further include an electronic display device 904 (e.g., for displaying graphics, text, and other data), a main memory 906 (e.g., random access memory (RAM)), storage device 908, a removable storage device 910 (e.g., removable storage drive, a removable memory module, a magnetic tape drive, an optical disk drive, a computer readable medium having stored therein computer software and/or data), user interface device 911 (e.g., keyboard, touch screen, keypad, pointing device), and a communication interface 912 (e.g., modem, a network interface (such as an Ethernet card), a communications port, or a PCM CIA slot and card). The communication interface 912 allows software and data to be transferred between the computer system and external devices. The system further includes a communications infrastructure 914 (e.g., a communications bus, cross-over bar, or network) to which the aforementioned devices/modules are connected as shown.

Information transferred via communications interface 914 may be in the form of signals such as electronic, electromagnetic, optical, or other signals capable of being received by communications interface 914, via a communication link 916 that carries signals and may be implemented using wire or cable, fiber optics, a phone line, a cellular/mobile phone link, an radio frequency (RF) link, and/or other communication channels. Computer program instructions representing the block diagram and/or flowcharts herein may be loaded onto a computer, programmable data processing apparatus, or processing devices to cause a series of operations performed thereon to produce a computer implemented process.

Embodiments have been described with reference to flowchart illustrations and/or block diagrams of methods, apparatus (systems) and computer program products according to embodiments. Each block of such illustrations/diagrams, or combinations thereof, can be implemented by computer program instructions. The computer program instructions when provided to a processor produce a machine, such that the instructions, which execute via the processor, create means for implementing the functions/operations specified in the flowchart and/or block diagram. Each block in the flowchart/block diagrams may represent a hardware and/or software module or logic, implementing embodiments. In alternative implementations, the functions noted in the blocks may occur out of the order noted in the figures, concurrently, etc.

Computer programs (i.e., computer control logic) are stored in main memory and/or secondary memory. Computer programs may also be received via a communications interface 912. Such computer programs, when executed, enable the computer system to perform the features of the embodiments as discussed herein. In particular, the computer programs, when executed, enable the processor and/or multi-core processor to perform the features of the computer system. Such computer programs represent controllers of the computer system.

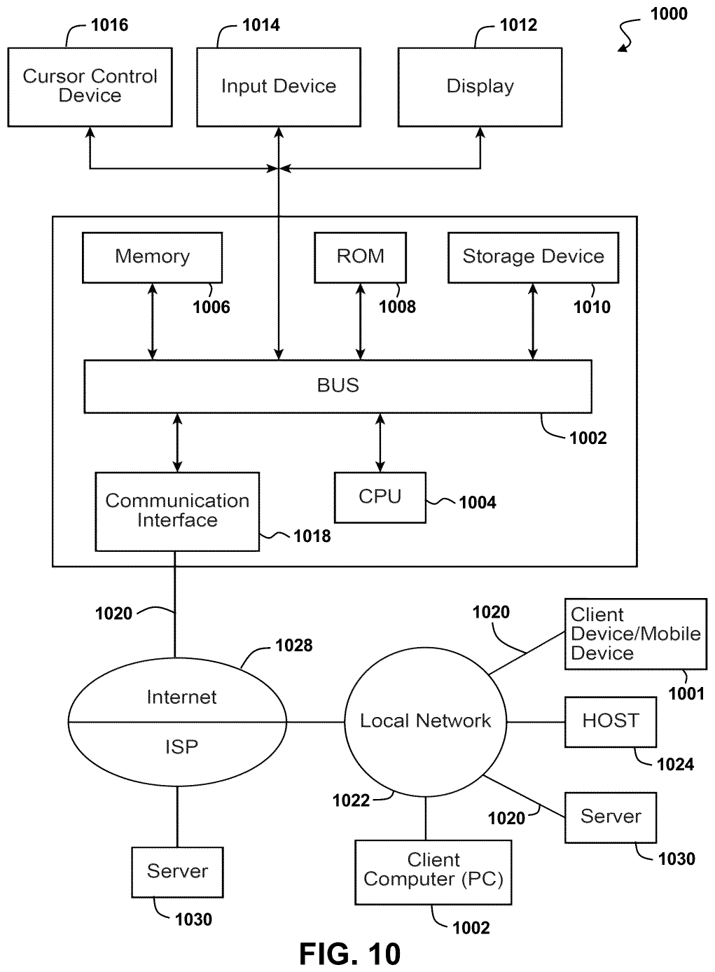

FIG. 10 shows a block diagram of an example system 1000 in which an embodiment may be implemented. The system 1000 includes one or more client devices 1001 such as consumer electronics devices, connected to one or more server computing systems 1030. A server 1030 includes a bus 1002 or other communication mechanism for communicating information, and a processor (CPU) 1004 coupled with the bus 1002 for processing information. The server 1030 also includes a main memory 1006, such as a random access memory (RAM) or other dynamic storage device, coupled to the bus 1002 for storing information and instructions to be executed by the processor 1004. The main memory 1006 also may be used for storing temporary variables or other intermediate information during execution or instructions to be executed by the processor 1004. The server computer system 1030 further includes a read only memory (ROM) 1008 or other static storage device coupled to the bus 1002 for storing static information and instructions for the processor 1004. A storage device 1010, such as a magnetic disk or optical disk, is provided and coupled to the bus 1002 for storing information and instructions. The bus 1002 may contain, for example, thirty-two address lines for addressing video memory or main memory 1006. The bus 1002 can also include, for example, a 32-bit data bus for transferring data between and among the components, such as the CPU 1004, the main memory 1006, video memory and the storage 1010. Alternatively, multiplex data/address lines may be used instead of separate data and address lines.

The server 1030 may be coupled via the bus 1002 to a display 1012 for displaying information to a computer user. An input device 1014, including alphanumeric and other keys, is coupled to the bus 1002 for communicating information and command selections to the processor 1004. Another type or user input device comprises cursor control 1016, such as a mouse, a trackball, or cursor direction keys for communicating direction information and command selections to the processor 1004 and for controlling cursor movement on the display 1012.

According to one embodiment, the functions are performed by the processor 1004 executing one or more sequences of one or more instructions contained in the main memory 1006. Such instructions may be read into the main memory 1006 from another computer-readable medium, such as the storage device 1010. Execution of the sequences of instructions contained in the main memory 1006 causes the processor 1004 to perform the process steps described herein. One or more processors in a multi-processing arrangement may also be employed to execute the sequences of instructions contained in the main memory 1006. In alternative embodiments, hard-wired circuitry may be used in place of or in combination with software instructions to implement the embodiments. Thus, embodiments are not limited to any specific combination of hardware circuitry and software.

The terms “computer program medium,” “computer usable medium,” “computer readable medium”, and “computer program product,” are used to generally refer to media such as main memory, secondary memory, removable storage drive, a hard disk installed in hard disk drive, and signals. These computer program products are means for providing software to the computer system. The computer readable medium allows the computer system to read data, instructions, messages or message packets, and other computer readable information from the computer readable medium. The computer readable medium, for example, may include non-volatile memory, such as a floppy disk, ROM, flash memory, disk drive memory, a CD-ROM, and other permanent storage. It is useful, for example, for transporting information, such as data and computer instructions, between computer systems. Furthermore, the computer readable medium may comprise computer readable information in a transitory state medium such as a network link and/or a network interface, including a wired network or a wireless network that allow a computer to read such computer readable information. Computer programs (also called computer control logic) are stored in main memory and/or secondary memory. Computer programs may also be received via a communications interface. Such computer programs, when executed, enable the computer system to perform the features of the embodiments as discussed herein. In particular, the computer programs, when executed, enable the processor multi-core processor to perform the features of the computer system. Accordingly, such computer programs represent controllers of the computer system.

Generally, the term “computer-readable medium” as used herein refers to any medium that participated in providing instructions to the processor 1004 for execution. Such a medium may take many forms, including but not limited to, non-volatile media, volatile media, and transmission media. Non-volatile media includes, for example, optical or magnetic disks, such as the storage device 1010. Volatile media includes dynamic memory, such as the main memory 1006. Transmission media includes coaxial cables, copper wire and fiber optics, including the wires that comprise the bus 1002. Transmission media can also take the form of acoustic or light waves, such as those generated during radio wave and infrared data communications.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, a PROM, an EPROM, a FLASH-EPROM, any other memory chip or cartridge, a carrier wave as described hereinafter, or any other medium from which a computer can read.

Various forms of computer readable media may be involved in carrying one or more sequences of one or more instructions to the processor 1004 for execution. For example, the instructions may initially be carried on a magnetic disk of a remote computer. The remote computer can load the instructions into its dynamic memory and send the instructions over a telephone line using a modem. A modem local to the server 1030 can receive the data on the telephone line and use an infrared transmitter to convert the data to an infrared signal. An infrared detector coupled to the bus 1002 can receive the data carried in the infrared signal and place the data on the bus 1002. The bus 1002 carries the data to the main memory 1006, from which the processor 1004 retrieves and executes the instructions. The instructions received from the main memory 1006 may optionally be stored on the storage device 1010 either before or after execution by the processor 1004.

The server 1030 also includes a communication interface 1018 coupled to the bus 1002. The communication interface 1018 provides a two-way data communication coupling to a network link 1020 that is connected to the worldwide packet data communication network now commonly referred to as the Internet 1028. The Internet 1028 uses electrical, electromagnetic or optical signals that carry digital data streams. The signals through the various networks and the signals on the network link 1020 and through the communication interface 1018, which carry the digital data to and from the server 1030, are exemplary forms or carrier waves transporting the information.

In another embodiment of the server 1030, interface 1018 is connected to a network 1022 via a communication link 1020. For example, the communication interface 1018 may be an integrated services digital network (ISDN) card or a modem to provide a data communication connection to a corresponding type of telephone line, which can comprise part of the network link 1020. As another example, the communication interface 1018 may be a local area network (LAN) card to provide a data communication connection to a compatible LAN. Wireless links may also be implemented. In any such implementation, the communication interface 1018 sends and receives electrical electromagnetic or optical signals that carry digital data streams representing various types of information.

The network link 1020 typically provides data communication through one or more networks to other data devices. For example, the network link 1020 may provide a connection through the local network 1022 to a host computer 1024 or to data equipment operated by an Internet Service Provider (ISP). The ISP in turn provides data communication services through the Internet 1028. The local network 1022 and the Internet 1028 both use electrical, electromagnetic or optical signals that carry digital data streams. The signals through the various networks and the signals on the network link 1020 and through the communication interface 1018, which carry the digital data to and from the server 1030, are exemplary forms or carrier waves transporting the information.

The server 1030 can send/receive messages and data, including e-mail, program code, through the network, the network link 1020 and the communication interface 1018. Further, the communication interface 1018 can comprise a USB/Tuner and the network link 1020 may be an antenna or cable for connecting the server 1030 to a cable provider, satellite provider or other terrestrial transmission system for receiving messages, data and program code from another source.

The example versions of the embodiments described herein may be implemented as logical operations in a distributed processing system such as the system 1000 including the servers 1030. The logical operations of the embodiments may be implemented as a sequence of steps executing in the server 1030, and as interconnected machine modules within the system 1000. The implementation is a matter of choice and can depend on performance of the system 1000 implementing the embodiments. As such, the logical operations constituting said example versions of the embodiments are referred to for e.g., as operations, steps or modules.

Similar to a server 1030 described above, a client device 1001 can include a processor, memory, storage device, display, input device and communication interface (e.g., e-mail interface) for connecting the client device to the Internet 1028, the ISP, or LAN 1022, for communication with the servers 1030.

The system 1000 can further include computers (e.g., personal computers, computing nodes) 1005 operating in the same manner as client devices 1001, where a user can utilize one or more computers 1005 to manage data in the server 1030.

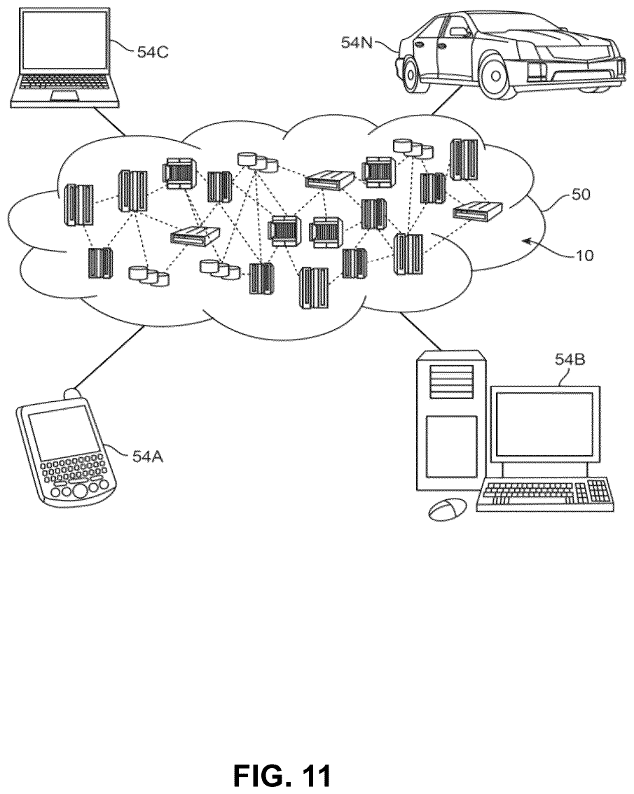

Referring now to FIG. 11, illustrative cloud computing environment 50 is depicted. As shown, cloud computing environment 50 comprises one or more cloud computing nodes 10 with which local computing devices used by cloud consumers, such as, for example, personal digital assistant (PDA), smartphone, smart watch, set-top box, video game system, tablet, mobile computing device, or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 11 are intended to be illustrative only and that computing nodes 10 and cloud computing environment 50 can communicate with any type of computerized device over any type of network and/or network addressable connection (e.g., using a web browser).

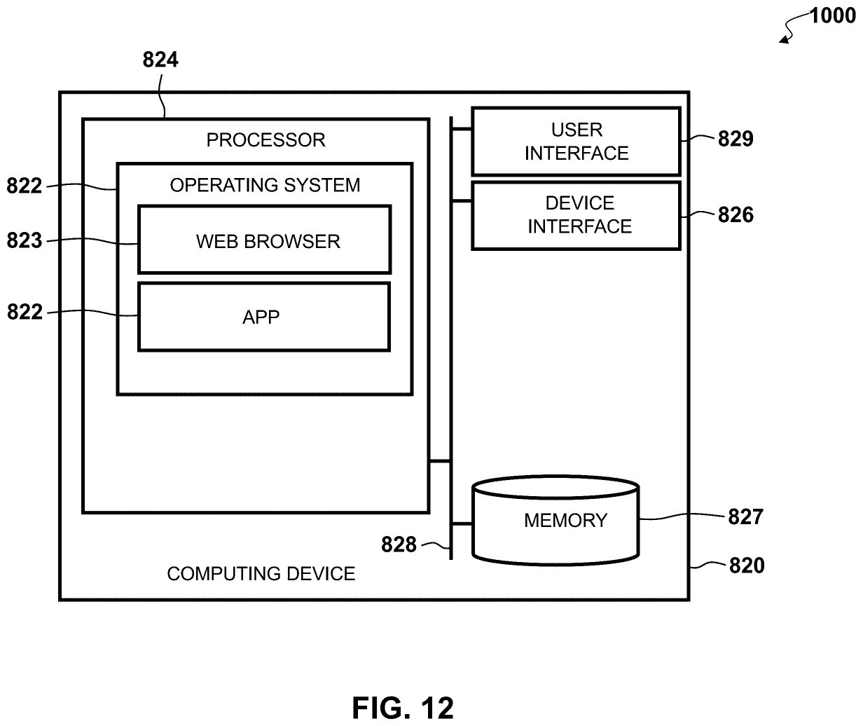

FIG. 12 illustrates an example of a top-level functional block diagram of a computing device embodiment 1200. The example operating environment is shown as a computing device 820 comprising a processor 824, such as a central processing unit (CPU), addressable memory 827, an external device interface 826, e.g., an optional universal serial bus port and related processing, and/or an Ethernet port and related processing, and an optional user interface 829, e.g., an array of status lights and one or more toggle switches, and/or a display, and/or a keyboard and/or a pointer-mouse system and/or a touch screen. Optionally, the addressable memory may include any type of computer-readable media that can store data accessible by the computing device 820, such as magnetic hard and floppy disk drives, optical disk drives, magnetic cassettes, tape drives, flash memory cards, digital video disks (DVDs), Bernoulli cartridges, RAMs, ROMs, smart cards, etc. Indeed, any medium for storing or transmitting computer-readable instructions and data may be employed, including a connection port to or node on a network, such as a LAN, WAN, or the Internet. These elements may be in communication with one another via a data bus 828. In some embodiments, via an operating system 825 such as one supporting a web browser 823 and applications 822, the processor 824 may be configured to execute steps of a process establishing a communication channel and processing according to the embodiments described above.

System embodiments include computing devices such as a server computing device, a buyer computing device, and a seller computing device, each comprising a processor and addressable memory and in electronic communication with each other. The embodiments provide a server computing device that may be configured to: register one or more buyer computing devices and associate each buyer computing device with a buyer profile; register one or more seller computing devices and associate each seller computing device with a seller profile; determine search results of one or more registered buyer computing devices matching one or more buyer criteria via a seller search component. The service computing device may then transmit a message from the registered seller computing device to a registered buyer computing device from the determined search results and provide access to the registered buyer computing device of a property from the one or more properties of the registered seller via a remote access component based on the transmitted message and the associated buyer computing device; and track movement of the registered buyer computing device in the accessed property via a viewer tracking component. Accordingly, the system may facilitate the tracking of buyers by the system and sellers once they are on the property and aid in the seller's search for finding buyers for their property. The figures described below provide more details about the implementation of the devices and how they may interact with each other using the disclosed technology.

It is contemplated that various combinations and/or sub-combinations of the specific features and aspects of the above embodiments may be made and still fall within the scope of the invention. Accordingly, it should be understood that various features and aspects of the disclosed embodiments may be combined with or substituted for one another in order to form varying modes of the disclosed invention. Further, it is intended that the scope of the present invention is herein disclosed by way of examples and should not be limited by the particular disclosed embodiments described above.

What is claimed is:

1. A method comprising:
scanning, by a camera module in communication with an application installed on a user device, a relevant part of a face, head, or body of a user;
mapping, by a computer vision module of the application, the scanned relevant part onto a mesh model to ensure consistent positioning, wherein the consistent positioning comprises whether the user device is being held upright using a combination of built-in motion and orientation sensors;
positioning, by the computer vision module, the scanned relevant part in a correct position based on the mesh model, wherein the correct position comprises having a facial positioning in relation to the camera module that is at an angle that is less than a predetermined threshold;
detecting, by a feature detection module of the application, a set of features associated with the scanned relevant part using the mesh model based on the scanned relevant part positioned in a correct position;
determining, by an image classification module of the application, a treatment area that is needed to be treated based on the detected features associated with the scanned relevant part;
capturing, by the camera module, an image of the relevant part based on the detected set of features;
transmitting, by the camera module, the captured image to a treatment option determination module of the application;
determining, by the treatment option determination module, at least one treatment option for the treatment area based on the transmitted captured image;
simulating, by an image-to-image translation module of the application, a treatment outcome on the treatment area for each of the determined at least one treatment option, producing a simulated image of the treatment area treated with the determined at least one treatment option; and
displaying, by a display module of the user device, recommendation for the determined at least one treatment option with relevant information, the relevant information including the simulated image and at least one of: the type of medical product, the volume and amount of the medical product, and the type of medical procedure.

2. The method of claim 1 further comprising:
determining, by the treatment option determination module, using machine learning neural networks, at least one treatment option for the treatment area based on the captured image.

3. The method of claim 2 wherein the detecting is based on performing a detailed analysis to label specific facial areas.

4. The method of claim 2 wherein the detecting is based on performing a detailed analysis to label specific facial areas, wherein the specific facial areas comprise: lips, nose, and under-eye region.

5. The method of claim 4 wherein the detecting is further based on identifying and classifying different parts of the relevant part for determining a set of appropriate treatment areas.

6. The method of claim 2 wherein determining at least one treatment option is based on understanding characteristics and needs of each specific treatment area.

7. The method of claim 6 wherein the understanding characteristics is based on recognizing signs of aging, dryness, or volume loss.

8. A system comprising:
a camera module in communication with an application installed on a user device;
a computer vision module;
a feature detection module;
an image classification module;
a treatment option determination module;
an image-to-image translation module; and
a display module;
wherein the system is configured to:
scan, by the camera module, a relevant part of a face, head, or body of a user;
map, by the computer vision module, the scanned relevant part onto a mesh model to ensure consistent positioning, wherein the consistent positioning comprises whether the user device is held upright via a combination of built-in motion and orientation sensors;
position, by the computer vision module, the scanned relevant part in a correct position based on the mesh model, wherein the correct position comprises a facial positioning in relation to the camera module that is at an angle that is less than a predetermined threshold;
detect, by the feature detection module, a set of features associated with the scanned relevant part using the mesh model based on the scanned relevant part positioned in a correct position;
determine, by the image classification module, a treatment area that is needed to be treated based on the detected features associated with the scanned relevant part;
capture, by the camera module, an image of the relevant part based on the detected set of features;
transmit, by the camera module, the captured image to a treatment option determination module of the application;
determine, by the treatment option determination module, at least one treatment option for the treatment area based on the transmitted captured image;
simulate, by the image-to-image translation module of the application, a treatment outcome on the treatment area for each of the determined at least one treatment option, producing a simulated image of the treatment area treated with the determined at least one treatment option; and
display, by the display module of the user device, recommendation for the determined at least one treatment option with relevant information, wherein the relevant information includes the simulated image and at least one of: the type of medical product, the volume and amount of the medical product, and the type of medical procedure.

9. The system of claim 8 further configured to:
determine, by the treatment option determination module, using machine learning neural networks, at least one treatment option for the treatment area based on the captured image.

10. The system of claim 9 wherein the system is configured to detect based on performing a detailed analysis to label specific facial areas.

11. The system of claim 9 wherein the system is configured to detect based on performing a detailed analysis to label specific facial areas, wherein the specific facial areas comprise: lips, nose, and under-eye region.

12. The system of claim 11 wherein the system is configured to detect further based on identifying and classifying different parts of the relevant part to determine a set of appropriate treatment areas.

13. The system of claim 9 wherein the system is configured to determine at least one treatment option based on understanding characteristics and needs of each specific treatment area.

14. The system of claim 13 wherein the system is configured to understand characteristics based on recognizing signs of aging, dryness, or volume loss.
